(19) United States
(12) Patent Application Publication
(10) Pub. No.: US 2019/0314187 A1
(43) Pub. Date: Oct. 17, 2019

(54) MUSCLE OR JOINT SUPPORT ARTICLE WITH BUMP

(71) Applicant: 3M INNOVATIVE PROPERTIES COMPANY, St. Paul, MN (US)

(72) Inventors: Diane L. Emslander, City of Grant, MN (US); Dominic J. Julian, Woodbury, MN (US); Jeffrey O. Emslander, City of Grant, MN (US)

(21) Appl. No.: 16/465,809

(22) PCT Filed: Nov. 29, 2017
(86) PCT No.: PCT/US2017/063548

§ 371 Date: May 31, 2019

Related U.S. Application Data
(60) Provisional application No. 62/429,389, filed on Dec. 2, 2016.
(60) Provisional application No. 62/579,273, filed on Oct. 31, 2017.

(51) Int. Cl. A61F 5/30

(52) U.S. Cl. CPC A61F 5/30; A61F 2013/0028

(57) ABSTRACT

The present application generally relates to support articles and methods of making and using them. The support articles of the present disclosure provide compression and/or stabilization of sore joints, tendons, or muscles. In some embodiments, the support articles include a bump protruding from at least a portion of the backing of the article that can be placed directly adjacent to a sore or affected area to provide enhanced compression or relief.

### TECHNICAL FIELD

The present application generally relates to support articles and methods of making and using them.

### BACKGROUND

A strained or injured muscle, tendon, or joint not only causes pain and discomfort, but can make someone feel slower or weaker than normal, all of which can interfere with daily living activities. As such, maintaining healthy skeletal muscles is essential to keeping people moving at their best. Historically, those with injuries or discomfort were instructed to limit or eliminate movement of the injured muscle, tendon, or joint. Today we know that, generally, keeping muscles moving improves circulation, can reduce pain, and/or speeds healing. Over the past twenty years, many innovative devices and techniques have come out of kinesiology science (the science behind muscular and skeletal movement) to help protect and treat injured or sore muscles, tendons, and joints.

Compression wraps, sleeves, and braces offer stabilization of the muscles, tendons, and joints covered by the wraps, sleeves, and braces. Additionally or alternatively, compression wraps, sleeves, and braces help to reduce pain, swelling, and cramping by increasing circulation and/or reducing fatigue. More specifically, an injured muscle often swells as a result of a build-up of fluid. The fluid build-up can prevent blood from circulating to the injured area, which results in slowing the healing process. Compression therapy generally uses a snug-fitting device to put pressure on the impacted area. The pressure reduces swelling and/or increases circulation by forcing fluid in the impacted area to move back into the blood vessels. As a result, the swelling decreases. Once the swelling has decreased, blood flow may return to the area and promote healing.

Kinesiology tapes (KT tapes) gently lift the skin and tissue attached to an injured muscle so that blood and other body fluids can move more freely in and around that muscle. Additionally, KT tapes can create neuromuscular feedback (called proprioception) that inhibits (relaxes) or facilitates stronger firing of muscles and tendons. This neuromuscular feedback can assist in support of the area without the bulk and restriction commonly associated with wraps and heavy bracing.

### SUMMARY

The inventors of the present disclosure recognized that existing commercial compression and kinesiology tape offerings have some disadvantages. For example, braces and compression sleeves, while highly effective, are bulky and can be challenging to wear discreetly under clothing. Further, braces and compression sleeves can be a significant monetary investment, and/or can get dirty and sweaty during prolonged use. Compression tapes and/or wraps can be challenging for non-professionally trained users to correctly apply at the correct tightness, potentially limiting their effectiveness and/or causing pain or harm. Kinesiology tapes provide good neuromuscular feedback but do not provide significant compression. Further, their ability to provide stabilization is somewhat limited.

The inventors of the present disclosure sought to create disposable and relatively inexpensive support articles that offer the best features of braces, compression sleeves, compression wraps, and kinesiology tapes while eliminating or minimizing their drawbacks. The inventors of the present disclosure sought to create support articles that offer muscle, tendon, or joint stabilization similar to a brace; muscle, tendon, or joint compression similar to a compression wrap; and the slim, discreet profile and neuromuscular feedback similar to kinesiology tape. In some embodiments, the support articles include a bump, bumper, or projection (referred to herein as a bump) that extends from the backing toward the affected area of the user and provides targeted, localized contact and/or pressure to the affected area. In some embodiments, the bump, bumper, or projection is a 3M Bumpon™ device.

The support articles of the present disclosure provide compression and/or stabilization of sore joints, tendons, or muscles. In some embodiments, the support article is a patch that, when applied, eliminates or reduces joint or muscle pain, soreness, and/or swelling and/or increases mobility and/or stability. In some embodiments, the support articles include a bump, bumper, or projection that extends from the backing toward the affected area of the user and provides targeted, localized contact and/or pressure to the affected area. In some embodiments, the support article additionally includes one or more reinforcing portions. In some embodiments, the support article also includes a strap. In some embodiments, the support article is a patch, cover, sheet, or strip. In some embodiments, the support article is noncircumferential (in other words, it does not extend around the circumference of a body part such as, for example, an ankle, leg, arm, etc. in the way that an elastic bandage (e.g., Ace™ bandage), elastic wrap, or compression roll would extend around the circumference of the body part). In some embodiments, the support article can easily be applied by a non-trained user and/or can be worn in many conditions (including, for example, in the shower or during exercise) for up to three days. In some embodiments, the support article has a relatively slim profile and are thus discreet such that they can be worn under clothing without being noticeable. Some support articles of the present disclosure are disposable after use. Further, in some embodiments, the disposable support articles cost significantly less than a brace or compression sleeve. In some embodiments, the support articles eliminate or reduce joint or muscle pain or soreness. In some embodiments, the support articles have a shape and size tailored for a specific body area. In some embodiments, the support articles are easy to apply and remove easily and/or without pain.

Some embodiments relate to a support article, comprising: a backing having a front major surface and a rear major surface; an adhesive adjacent to or included in at least a portion of the rear major surface of the backing layer; and a bump that extends or protrudes from the rear major surface of the backing.

Any of the embodiments described herein can include a release liner adjacent to at least a portion of the adhesive. Any of the embodiments described herein can include a backing including at least one of a polyurethane film, a polyethylene film, a polypropylene film, a PVC film, a nonwoven material, and/or a woven material. Any of the embodiments described herein can include a backing including at least one of a polyolefin, polyester, polyalkylene, polyamide, polystyrene, polyarylsulfone, polydiene, and/or polyurethane. Any of the embodiments described herein can include a backing including conjugate multicomponent melt spun fibers. Any of the embodiments described herein can include a backing having a weight of between about 25 gsm to about 300 gsm. Any of the embodiments described herein can include the backing having a thickness of about 0.01 cm to about 1 cm.

Any of the embodiments described herein can include at least one of the backing or the backing and adhesive combination having a breathability and/porosity of between about 3 and about 12 mm H₂O measured using the pressure drop test. Any of the embodiments described herein can include at least one of the backing or the backing and adhesive combination having a cross-directional tensile strength of between about 4 lbf (17.8 N) and about 9 lbf (40.0 N) and/or a machine-directional tensile strength of between about 5 lbf (22.2 N) and about 10 lbf (44.5 N). Any of the embodiments described herein can include at least one of the backing or the backing and adhesive combination having a cross-directional elongation at break of between about 600% and about 900% and/or a machine-directional elongation at break of between about 350% and about 1000%. Any of the embodiments described herein can include the backing and adhesive forming a conjugate multicomponent system.

Any of the embodiments described herein can include the adhesive being a pressure sensitive adhesive and being selected from at least one of the following adhesive classes: polyacrylate adhesives, polyalphaolefin adhesives, polyvinyl acrylates, rubber resin adhesives, silicone adhesives, polydiorganosiloxane polyurea compolymers, and mixtures thereof.

Any of the embodiments described herein can include one or more reinforcing portions. Any of the embodiments described herein can include the reinforcing portion(s) covering between about 10% and about 75% of the total surface area of the backing. Any of the embodiments described herein can include the adhesive adhering the one or more reinforcing portions to the front or rear major surface of the backing. Any of the embodiments described herein can include the one or more reinforcing portions including at least one of foam or a shaped memory material. In some embodiments where the support article includes a reinforcing portion, one or more bumps may be in contact only with the reinforcing portion but not the backing. Any of the embodiments described herein can include the one or more reinforcing portions having a Shore A hardness is between about 10 and about 100 when measured according to ASTM D2240 and/or a Shore D hardness of between about 10 and about 60 when measured according to ASTM D2240. Any of the embodiments described herein can include the one or more reinforcing portions having a cross-directional tensile strength of between about 13 lbf (57.8 N) and about 28 lbf (129.0 N) and/or a machine-directional tensile strength of between about 16 lbf (71.2 N) and about 31 lbf (137.9 N). Any of the embodiments described herein can include the one or more reinforcing portions having a thickness of between about 2 cm (787 mil) and about 0.051 cm (20 mil). Any of the embodiments described herein can include the one or more reinforcing portions having an elongation at break of between about 10% and about 50%. Any of the embodiments described herein can include at least two reinforcing layers that are on layered on one another. Any of the embodiments described herein can include at least one of the reinforcing portions being separate from the support article and being independently applied by the user before the full support article is applied.

Any of the embodiments described herein can include the one or more reinforcing portions comprising: an adhesive layer having first and second major surfaces; a first foam layer adjacent to first major surface of the adhesive layer; a second foam layer adjacent to second major surface of the adhesive layer; a first skin layer adjacent to first foam layer; and a second skin layer adjacent to second foam layer. Any of the embodiments described herein can include microspheres in at least one of the first or second foam layers.

Any of the embodiments described herein can also include a strap a strap attached or adhered to the front major surface of the backing, the strap being capable of stretching across at least a portion of the support article. In some such embodiments, the strap is repositionable. In some embodiments, the strap includes at least one of a polyolefin, a modified polyolefin, a polyvinyl chloride, a polycarbonate, polystyrene, polyester, polylactide, polyvinylidene fluoride, (meth)acrylic, urethane, acrylic urethane, ethylene vinyl acetate copolymer, acrylate-modified ethylene vinyl acetate polymer, ethylene acrylic acid copolymers, nylon, engineering polymer, or elastomer.

Any of the embodiments described herein can include a support article applied to or sized for application to at least one of the IT band, hip, calf, shin, quads, hamstrings, groin, hip flexor, gluteus, outer knee, inner knee, Osgood shlatter, back of knee, front of knee, Achilles tendon, ankle, ball of foot, top of foot, heel, toe, finger, SI joint, low back, middle back, ribs, spine, abdominal, neck, shoulder, rotator cuff, AC joint, wrist, elbow, thumb, bicep, and/or tricep.

### BRIEF DESCRIPTION OF DRAWINGS

FIG. 1 is a front (or top) view of one exemplary embodiment of a support article in accordance with the teachings of the present disclosure.

FIGS. 2A-2C are rear (or back or bottom) views of three different exemplary embodiments of a support article whose front major surface is shown in FIG. 1.

FIG. 3 is a schematic drawing of the support article of FIGS. 1-2 in use.

FIGS. 4A-4D show various views of some differing embodiments of support article embodiments in accordance with teachings herein. FIG. 4A is a front (or top) view of one exemplary embodiment of a support article in accordance with the teachings of the present disclosure. FIGS. 4B and 4C are rear (or back or bottom) views of two different embodiments of support articles whose front (or top) major surface is shown in FIG. 4A. FIG. 4D shows the embodiment of FIG. 4B in use.

FIG. 5A is a front (or top) view of an exemplary support article in accordance with the teachings herein.

FIG. 5B is a rear (or back or bottom) view of one exemplary embodiment of the support article shown in FIG. 5A.

FIG. 5C is a schematic drawing of the support article of FIGS. 5A and 5B in use and/or during application.

FIG. 6A is a front (or top) view of an exemplary support article in accordance with the teachings herein.

FIG. 6B is a schematic drawing the support article of FIG. 6A in use and/or during application.

FIGS. 7-12 are schematic drawings of six exemplary bumps that may be used in connection with the support articles described herein.

FIG. 13 is a cross-sectional view of an exemplary reinforcing portion.

In the following detailed description, reference may be made to the above-described set of drawings in which are shown by way of illustration several exemplary embodiments. It is to be understood that other embodiments are contemplated and may be made without departing from the scope or spirit of the present disclosure.

### DETAILED DESCRIPTION

Various embodiments and implementations will be described in detail. These embodiments should not be construed as limiting the scope of the present application in any manner, and changes and modifications may be made without departing from the spirit and scope of the inventions described herein. Further, only some end uses have been discussed herein, but end uses not specifically described herein are included within the scope of the present application. For example, the support article can be referred to as a patch. A patch is merely one exemplary embodiment. As used herein, the term “patch” refers to a piece of material used to cover an injured or sensitive area of the body. The patch preferably has adherent properties such that it can adhere to the injured or sensitive area of the body. The term patch does not require the presence of a medicament, although, as is described in more detail herein, the embodiments described herein could include such a medicament. As such, the scope of the present application should be determined by the claims.

The present disclosure generally relates to support articles. In some embodiments, when applied, the support article eliminates or reduces joint or muscle pain or soreness and/or increases mobility and/or stability. In some embodiments, these benefits are provided, at least in part, by a bump (a bump, bumper, or projection) that extends from the backing toward the affected area of the user and provides targeted, localized contact and/or pressure to the affected area when positioned adjacent to the affected or sensitive area of the user during application and/or use. In some embodiments, the application of point pressure (supplied by the bump) on or proximally to the pain point can help to provide counterforce below the tendon insertion point. This counterforce can relieve the concentration of forces at the tendon insertion point thereby relieving associated pain and discomfort from, for example, tendinitis.

Some embodiments include a strap that extends across a portion of the support article and increases the compression force of the bump, reinforcing portions, and/or support article on the user and/or affected area.

In some embodiments, the support article has a shape and size tailored for a specific body area. In some embodiments, the support article can be used in any of the following exemplary body areas: IT band, hip, calf, shin, quads, hamstrings, groin, hip flexor, gluteus, outer knee, inner knee, Osgood shlatter, back of knee, front of knee, Achilles tendon, ankle, ball of foot, top of foot, heel, toe, finger, SI joint, low back, middle back, ribs, spine, abdominal, neck, shoulder, rotator cuff, AC joint, wrist, elbow, thumb, bicep, and/or tricep. In some embodiments, the support article has a more general use and a single shape or design can be used in a number of different body areas.

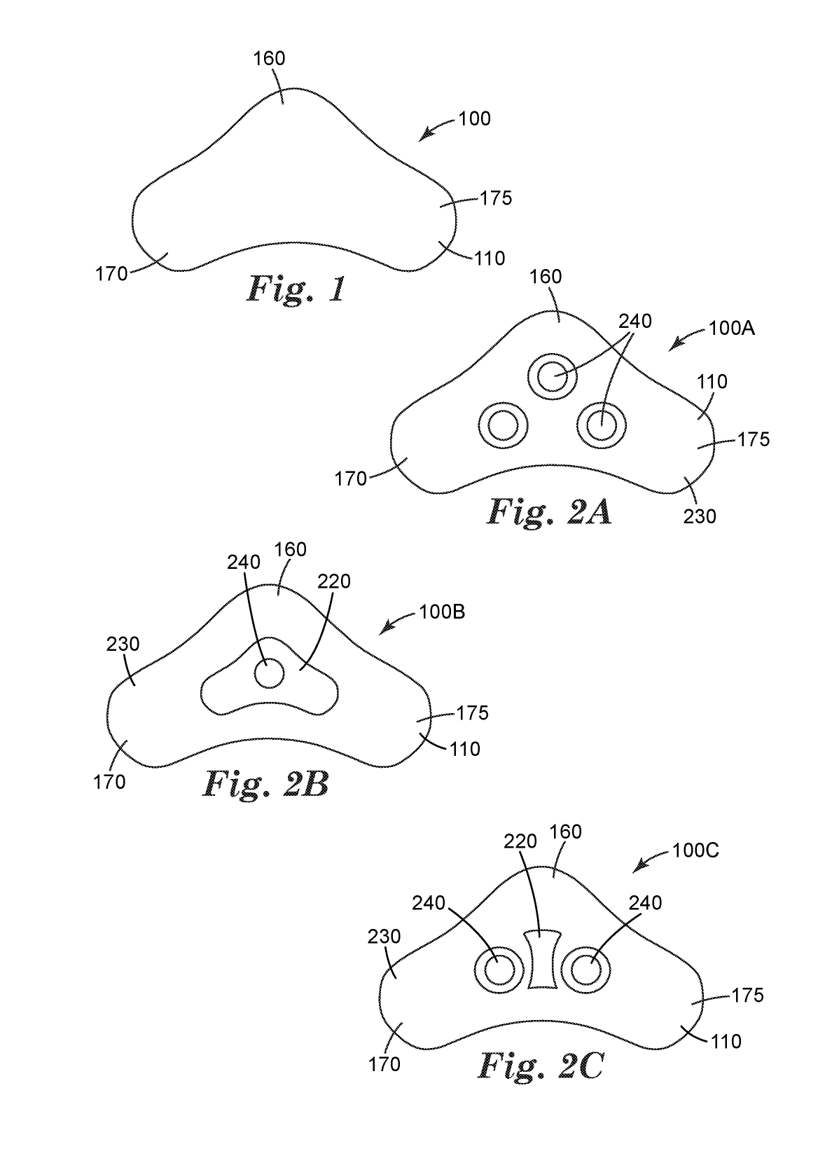

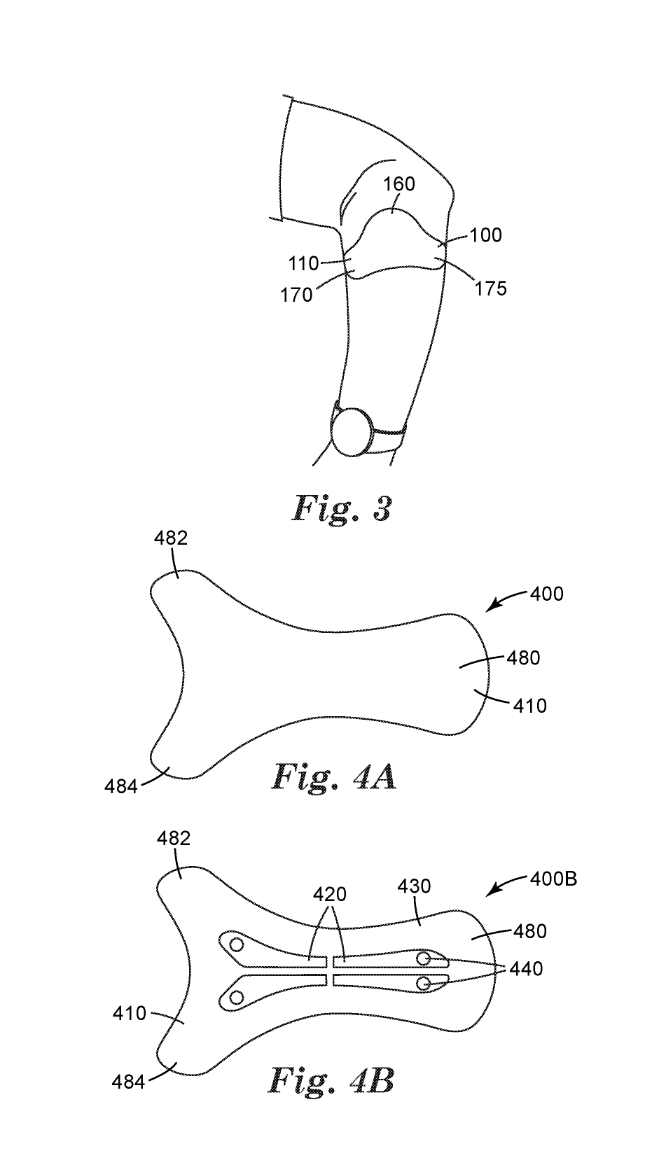

FIGS. 1-3 show various support article embodiments that can be used, for example, as elbow support articles (as shown more specifically in FIG. 3). FIG. 1 is a front (or top) view of one exemplary embodiment of a support article 100 in accordance with the teachings of the present disclosure. Support article 100 includes a backing in the shape generally shown. The front (or top) major surface of backing 110 is shown in FIG. 1.

FIGS. 2A-2C are rear views of three exemplary embodiments of support articles having the front surface shown in FIG. 1. In all embodiments shown in FIGS. 2A, 2B, and 2C, back (or rear or bottom) major surface of backing 110 is at least partially coated with or adjacent to an adhesive 230. In the specific embodiment shown in FIG. 2A, support article 100A includes three bumps 240 that are adhered, attached, or adjacent to the back (or rear or bottom) major surface of backing 110. In this specific embodiment, there are three bumps 240 that are each identically or similarly shaped and sized. However, those of skill in the art will appreciate that any number, shape, size, or thickness bump or reinforcing portion may be used. In the specific embodiment shown in FIG. 2B, support article 100B includes a reinforcing portion 220 adjacent to rear (or back or bottom) major surface of backing 110. A bump 240 is adhered, attached, or adjacent to reinforcing portion 220. Those of skill in the art will appreciate that any number, shape, size, or thickness bump may be used. In the specific embodiment shown in FIG. 2C, support article 100C includes a reinforcing portion 220 adjacent to rear (or back or bottom) major surface of backing 110. Support article 100C also includes two bumps 240 adhered, attached, or adjacent to rear (or back or bottom) major surface of backing 110. Those of skill in the art will appreciate that any number, shape, size, or thickness bump or reinforcing portion may be used.

FIG. 3 is a schematic drawing of the support article of the type generally shown in any of FIGS. 1-3 in use on a human's elbow.

The elbow support embodiments shown in FIGS. 1-3 are merely exemplary and may be modified in any way described herein and/or obvious to those of skill in the art. For example, any desired number, shape, size, or thickness reinforcing portion may be used. Reinforcing portion(s) are optional and need not be included. Any desired number, shape, size, or thickness bump, projection, or bumper may be used. Any desired shape, size, or thickness backing may be used. Any of the backings, adhesives, bumps, and/or reinforcing portions described herein can be used. Any adhesive capable of use on skin can be used, as is discussed in greater detail herein. The reinforcing portions can be adhesively attached or adhered to the backing or can be mechanically attached or adhered to the backing, as is described in greater detail herein. The bump(s) can be adhesively attached or adhered to the backing or reinforcing portion or can be mechanically attached or adhered to the backing or reinforcing portion, as is described in greater detail herein. An optional release liner (not shown) may be positioned adjacent to at least a portion of the adhesive and/or backing. The release liner may extend over the reinforcing portions or may have a cut out around reinforcing portion(s) and/or bump(s). The support article positioning on the body may differ from that shown.

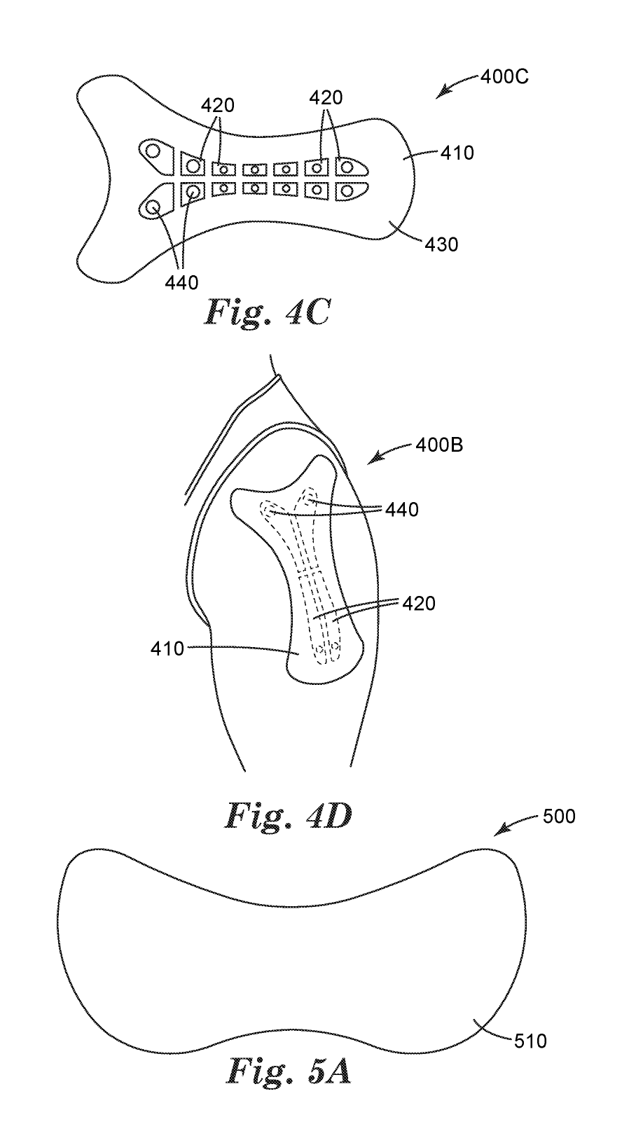

FIGS. 4A-4D show various views of some differing embodiments of support article embodiments that can be used, for example, as shoulder support articles (as shown more specifically in FIG. 4D). FIG. 4A is a top (or front) view of one exemplary embodiment of a support article in accordance with the teachings of the present disclosure. FIG. 4A is a front (or top) view of support article 400. Support article 400 includes a backing 410, the front (or top) major surface of which is shown in FIG. 4A. Backing 410 has the shape generally shown.

FIGS. 4B and 4C are rear views of two different embodiments of support articles whose front (or top) major surface looks like that shown in FIG. 4A. FIGS. 4B and 4C are rear (or back or bottom) views of two different embodiments of support articles whose front (or top) major surface is shown in FIG. 4A.

FIG. 4B shows a rear (or back or bottom) view of an exemplary embodiment of a support article 400B. The rear (or back or bottom) major surface of backing 410 is at least partially coated with or adjacent to adhesive 430. The specific embodiment shown in FIG. 4B includes four reinforcing portions 420 on each of which is positioned a generally circular shaped bump 440. The one or more bumps 440 can be adhered to backing 410, reinforcing portions 420, or adhesive 430 or can be mechanically attached thereto by, for example, the methods described herein. The user can position the one or more bumps 440 adjacent to a sore or affected area to provide targeted, point pressure and/or compression. Reinforcing portions 420 simultaneously provide support to the area adjacent to the affected area.

FIG. 4C shows a rear (or back or bottom) view of an exemplary embodiment of a support article 400C. The rear (or back or bottom) major surface of backing 410 is at least partially coated with or adjacent to adhesive 430. The specific embodiment shown in FIG. 4C includes fourteen reinforcing portions 420 on each of which is positioned a generally circular shaped bump 440. The one or more bumps 440 can be adhered to backing 410, reinforcing portions 420, or adhesive 430 or can be mechanically attached thereto by, for example, the methods described herein. The user can position the one or more bumps 440 adjacent to a sore or affected area to provide targeted, point pressure and/or compression. Reinforcing portions 420 simultaneously provide support to the area adjacent to the affected area.

FIG. 4D is a schematic drawing of support article 400B in use on a human's shoulder. Reinforcing portions 420 and bumps 440 are shown in dashed lines because they are on the underside of the backing and, as such, are not visible once the support article is applied.

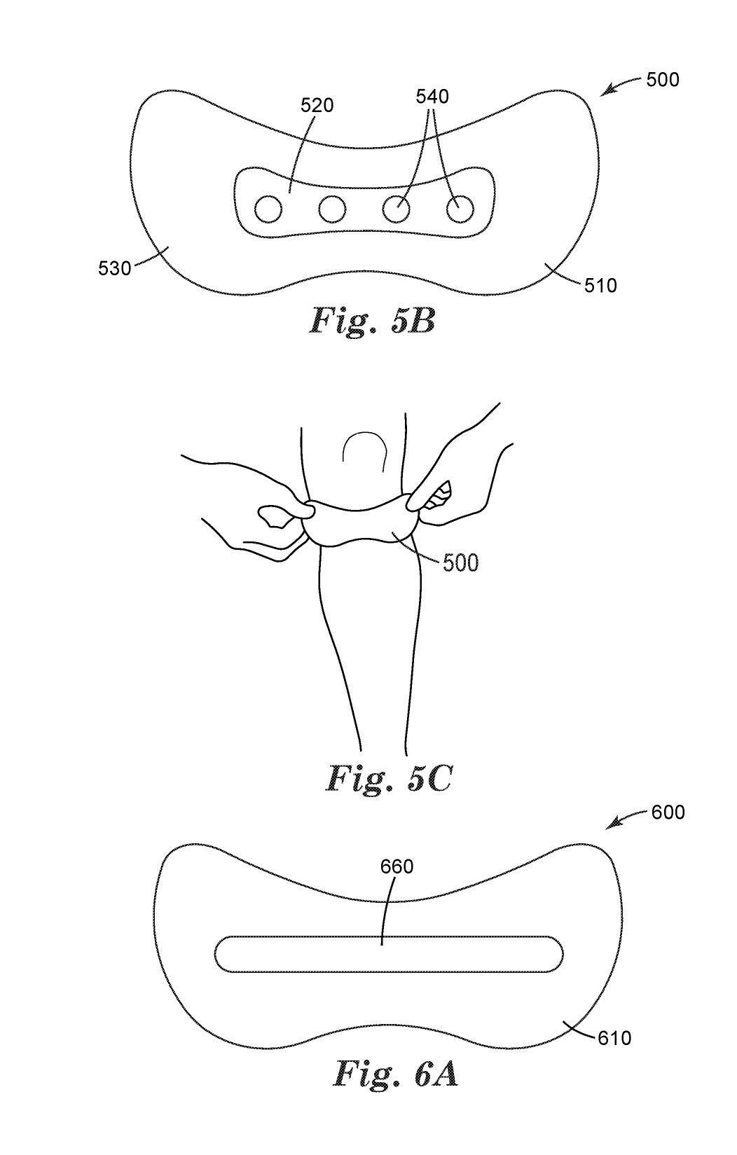

FIGS. 5A-5C show various views of an exemplary embodiments of a support article that can be used, for example, as knee support articles. FIG. 5A is a front (or top) view of an exemplary support article 500. FIG. 5A shows the front (or top) major surface of a backing 510 which has the general shape shown. FIG. 5B is a rear (or back or bottom) view of support article 500 and shows a rear (or back or bottom) major surface of backing 510. Adhered, attached, or adjacent to backing 510 is an adhesive 530. In the specific implementation shown in FIG. 5B, four spaced apart bumps 540 are adhered, attached, or adjacent to a single reinforcing portion 520. FIG. 5C is a schematic drawing of support article 500 of FIGS. 5A and 5B in use and/or during application. The user is gripping or holding the terminal ends along the longitudinal length of support article 500 at their maximum length and then applying the support article such that adhesive 530 on the rear (or back or bottom) major surface of backing 510 contacts and sticks to the user's skin on his/her knee. This positions the reinforcing portion (where present) and/or bump 540 adjacent to or near the affected, swollen, and/or sore areas of the knee.

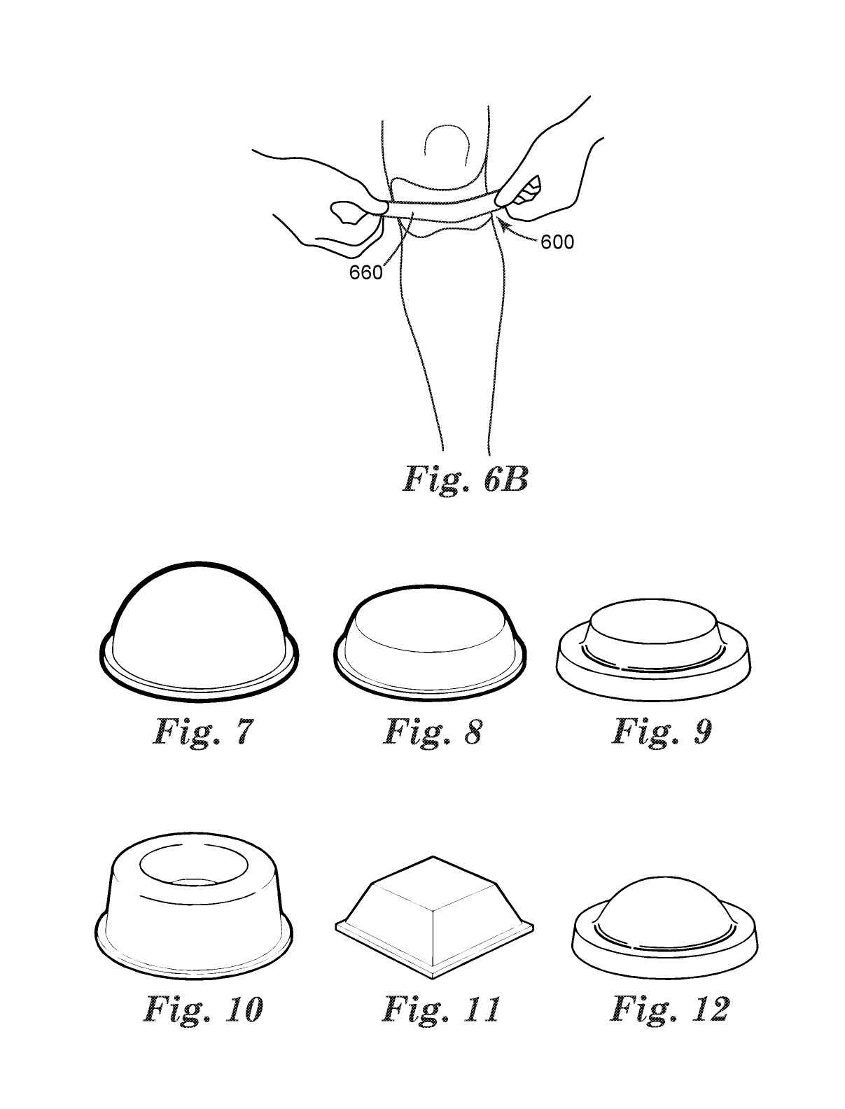

FIGS. 6A and 6B show front views of an exemplary embodiment of a support article that can be used, for example, as knee support article. FIG. 6A is a front (or top) view of an exemplary support article 600 in accordance with the teachings herein. Support article 600 is substantially the same as support article 500 of FIGS. 5A and 5B except that it includes a strap 640 on the front (or top) major surface of backing 610. Strap 660 extends across a portion of the front (or top) major surface of backing 610 of support article 600 and, when applied, increases the compression force of the support article on the affected area of the user. Any rear surface configuration for support article 600 covered by the teachings of the present disclosure can be used for support article 600.

FIG. 6B is a schematic drawing of support article 600 of FIG. 6A in use and/or during application. The user is gripping or holding the terminal ends of strap 660 along their longitudinal length and then applying the adhesive-coated, rear, skin-facing portion of strap 660 to one of backing 610 or to the user's skin (depending on the amount of pressure and/or compression the user wants to apply to the affected, sore, and/or swollen area). This positions the reinforcing portion (where present) and/or bump(s) adjacent to or near the affected, swollen, and/or sore areas of the knee while also providing enhanced compression and/or pressure due to activation of the strap.

Backing:

The backing can include any acceptable backing layer. The backing can be a single layer or multilayer. In some embodiments, the backing layer is a nonwoven layer. In some embodiments, the backing is at least one of a polyurethane film, a polyethylene film, a polypropylene film, a PVC film, an elastic nonwoven fabrics, and/or a woven material.

In some embodiments, the backing layer includes conjugate multicomponent melt spun fibers. For example, the backing layer can be any of the backing layers described in, for example, U.S. Pat. No. 6,107,219 (Joseph et al.), the entirety of which is incorporated by reference herein. The fibers used to form the nonwoven backing can be, for example, polymeric. In some embodiments, the fibers are organic polymeric materials. Some exemplary suitable materials for use in forming conjugate multicomponent fibers include polyolefins, polyesters, polyalkylenes, polyamides, polystyrenes, polyarylsulfones, polydienes or polyurethanes. These materials are preferably extensible or slightly elastomeric, but could be elastomeric. In some embodiments, extensible or slightly elastomeric polyurethanes may be preferred (e.g., “MORTHANE” PS 440-200 resin available from Morton Thiokol Corp; also known as “IROGRAN” PS440-200 from Huntsman); and polyolefins such as polyethylenes, polypropylenes, ethylene-propylene copolymers, ethylene/vinyl acetate copolymers, or metallocene-type polyethylenes having a density of greater than 0.87 grams/cm³. Other suitable elastomeric materials include metallocene-type polyethylene copolymers (apparent density less than 0.87 grams/cm³); polyolefin elastomers (e.g., ethylene/propylene/diene elastomers); A-B block copolymers, as described above, having A blocks formed of poly (vinyl arenes) such as polystyrene and B blocks formed of conjugated dienes such as isoprene, butadiene, or hydrogenated versions thereof (e.g., “KRATON” elastomers available from Kraton Co.); polyetheresters (such as “ARNITEL” available from DSM); or polyether block amides (such as “PEBAX” available from Atochem Co.). Blends of elastomers, blends of nonelastomers or blends of both elastomers and nonelastomers can also be used.

In some embodiments, fibers having a diameter of no greater than about 10 microns are formed. In some embodiments, fibers having a diameter up to about 50 microns or more can be prepared using a melt-blown process. In some embodiments, fibers having a diameter of up to about 100 microns can be prepared using a spun bond process.

The webs formed can be of any suitable thickness for the desired and intended end use. In some embodiments, the nonwoven backing has a thickness of about 0.01 cm to about 5 cm.

In some embodiments, the backing has a weight of between about 60 gsm to about 200 gsm. In some embodiments, the backing has a weight of between about 120 gsm to about 160 gsm.

In some embodiments, the backing has a cross-directional tensile strength of between about 4 lb and about 9 lb. In some embodiments, the backing has a cross-directional tensile strength of between about 5 lb and about 8 lb. In some embodiments, the backing has a cross-directional tensile strength of between about 6 lb and about 7 lb. In some embodiments, the backing has a cross-directional tensile strength of greater than about 4 lb. In some embodiments, the backing has a cross-directional tensile strength of greater than about 5 lb. In some embodiments, the backing has a cross-directional tensile strength of less than about 9 lb. In some embodiments, the backing has a cross-directional tensile strength of less than about 8 lb.

In some embodiments, the backing has a machine-directional tensile strength of between about 5 lb and about 10 lb. In some embodiments, the backing has a machine-directional tensile strength of between about 6 lb and about 9 lb. In some embodiments, the backing has a machine-directional tensile strength of between about 7 lb and about 8 lb. In some embodiments, the backing has a machine-directional tensile strength of greater than about 5 lb. In some embodiments, the backing has a machine-directional tensile strength of greater than about 6 lb. In some embodiments, the backing has a machine-directional tensile strength of less than about 10 lb. In some embodiments, the backing has a machine-directional tensile strength of less than about 9 lb.

Tensile strength can be measured by using a tensile tester (for example, one obtained under the trade designation “ZWICK” from Zwick USA, Kennesaw, Ga.). For each sample, test specimens can be cut so that testing could be performed in the downweb (or machine) direction and in the crossweb (or transverse) direction. Test specimens can be cut to be 1 inch (2.54 cm) in width. The gauge length can be 5.0 cm. Depending on the value being measured (e.g, downweb or crossweb tensile strength) clamps can be attached to downweb ends or crossweb ends of the specimen. Crosshead speed can be 20.0 cm/min. For both downweb and crossweb specimens, the maximum force achieved during the test, and the strain at the force maximum, was recorded.

In some embodiments, the backing has a cross-directional elongation of between about 600% to about 900%. In some embodiments, the backing has a cross-directional elongation of between about 650% and about 850%. In some embodiments, the backing has a cross-directional elongation of between about 600% and about 800%. In some embodiments, the backing has a cross-directional elongation of greater than about 600%. In some embodiments, the backing has a cross-directional elongation of greater than about 700%. In some embodiments, the backing has a cross-directional elongation of less than about 900%. In some embodiments, the backing has a cross-directional elongation of less than about 800%.

In some embodiments, the backing has a machine-directional elongation of between about 700% to about 1000%. In some embodiments, the backing has a machine-directional elongation of between about 750% and about 950%. In some embodiments, the backing has a machine-directional elongation of between about 700% and about 900%. In some embodiments, the backing has a machine-directional elongation of greater than about 700%. In some embodiments, the backing has a machine-directional elongation of greater than about 800%. In some embodiments, the backing has a machine-directional elongation of less than about 1000%. In some embodiments, the backing has a machine-directional elongation of less than about 900%.

In some embodiments, the nonwoven backing includes additional fibers, such as, for example, other melt spun fibers, staple fibers (including inorganic and organic fibers, such as thermoplastic fibers, carbon fibers, glass fibers, or mineral fibers), organic binder fibers, and/or fibers of different polymers. Alternatively, other polymer materials can be simultaneously melt processed with the multicomponent fibers of the present invention to form webs containing more than one type of melt processed fiber, preferably, melt blown microfiber. Webs having more than one type of fiber are referred to herein as having commingled constructions. In commingled constructions, the various types of fibers can be intimately mixed forming a substantially uniform cross-section, or they can be in separate layers. The web properties can be varied by the number of different fibers used, the number of layers or regions employed, and the layer or region arrangement. Other materials, such as surfactants or binders can also be incorporated into the web before, during, or after its collection, such as by the use of a spray jet.

In some embodiments, the fibers forming the nonwoven article backing are intimately entangled each with the other in the form of a coherent breathable fibrous nonwoven article backing.

Exemplary suitable nonwoven article backings can be formed as melt blown microfiber webs using the apparatus discussed, for example, in Wente, Van A., “Superfine Thermoplastic Fibers”, Industrial Engineering Chemistry, Vol. 48, pages 1342-1346, Wente, Van A. et al., “Manufacture of Superfine Organic Fibers”, Report No. 4364 of the Navel Research Laboratories, published May 25, 1954, and in U.S. Pat. Nos. 3,849,241; 3,825,379; and others. These microtine fibers are termed melt blown fibers or blown microfibers (BMF) and are generally substantially continuous and form into a coherent web between the exit die orifice and a collecting surface by entanglement of the microfibers due in part to the turbulent airstream in which the fibers are entrained. Other conventional melt spinning type processes, such as spunbond processes where the fibers are collected in a web form immediately upon fiber formation, can also be used to form the nonwoven article backing. In some embodiments, the fibers are 100 microns or less in diameter when formed by melt spinning type processes, preferably 50 microns or less. The multicomponent fibers, if formed by the melt blown process, can be produced as described in U.S. Pat. No. 5,176,952 (Joseph et al); U.S. Pat. No. 5,232,770 (Joseph); U.S. Pat. No. 5,238,733 (Joseph et al); U.S. Pat. No. 5,258,220 (Joseph); or U.S. Pat. No. 5,248,455 (Joseph et al), each of which is incorporated by reference herein in its entirety. The multicomponent fiber can also be produced by a spunbond process as are disclosed in U.S. Pat. No. 5,695,868 (McCormach); U.S. Pat. No. 5,336,552 (Strack et al); U.S. Pat. No. 5,545,464 (Stokes); U.S. Pat. Nos. 5,382,400; 5,512,358 (Shawyer et al); or U.S. Pat. No. 5,498,463 (McDowall et al), each of which is incorporated by reference herein in its entirety.

In some embodiments, the backing layer is breathable and/or porous. In some embodiments, the backing layer is highly breathable and/or porous, making the support article comfortable to wear and/or to minimize or prevent itching, irritation, or undesirable skin reactions. In some embodiments, the backing layer allows for moisture release. The more porous the backing layer, the better the backing layer will release moisture caused by sweating or being worn in water or humid environments. In some embodiments, the backing layer has a breathability and/porosity of between about 3 and about 12 mm H₂O measured using the pressure drop test. In some embodiments, the backing layer has a breathability and/porosity of between about 4 and about 12 mm H₂O measured using the pressure drop test. In some embodiments, the backing layer has a breathability and/porosity of at least about 3 mm H₂O measured using the pressure drop test. In some embodiments, the backing layer has a breathability of at least about 5 mm H₂O measured using the pressure drop test. As used herein, the pressure drop test involves cutting a 5.25 inch circle from a MBPU web; placing the sample into a penetrometer (such as, for example, Model #8810 AFT Penetrometer with a 4.5 inch elastomeric chuck) with the airflow at 85 liters per minute; the sample should be set in the penetrometer collector (smooth) side down onto the lower flat chuck of the test equipment and securing it in place with the upper chuck; and measuring the recording the pressure drop in mm H₂O.

In some embodiments, the backing has a basis weight of from 25 to 200 g/m²; however, the basis weight could be significantly higher with added particulates and/or fibers.

Adhesive

Adhesives used in the present disclosure can include at least two adhesives: (1) the adhesive used to adhere the support article to the user (located on the rear (or bottom or back) side of the backing); and (2) the adhesive (where present) used to adhere the reinforcing portion(s) bump(s), or strap(s) to the rear (or bottom or back) side of the backing. In some embodiments, these two adhesives are the same. In some embodiments, these two adhesives differ. In some embodiments, the reinforcing portion(s), bump(s), or strap(s) are attached to the backing using non-adhesive means, in which case the second adhesive would not be present. Some exemplary non-adhesive means include lamination, ultrasonic welding, hook and loop, etc. Each of these adhesives will be described in greater detail below.

Adhesives used to Adhere the Backing or Support Article to the User

Any adhesive capable of use on skin may be used on the rear (or back or bottom) side of the backing to adhere the backing to the user. Selection of a desired adhesive to adhere the backing to the user may be based on various factors including, for example, the region of the body on which the support article is meant to be used, the skin sensitivity profile of the end user, etc. Some exemplary adhesives include those described in, for example, U.S. Pat. No. 6,107,219 (Joseph et al.), U.S. Pat. No. 6,703,120 (Ko et al.); U.S. Pat. No. 7,407,709 (Zhou et al.); U.S. Pat. No. 7,807,268 (Zhou et al.); U.S. Pat. No. 9,359,529 (Liu et al.); U.S. Pat. No. 8,541,481 (Determan et al.); U.S. Pat. No. 9,017,771 (Determan et al.); U.S. Pat. No. 6,730,397 (Melancon et al.); U.S. Pat. No. 8,822,559 (Zoller et al.); and U.S. Pat. No. 8,822,560 (Seth et al.) and U.S. Patent Publication Nos. 2011-0206924 (Liu et al.), 2014-0220843 (Liu et al.), 2015-0165087 (Fung et al.), 2015-0376345 (Liu et al.), 2017/081573 (Kipke et al.), 2015/299542 (Determan et al.), 2013/040073 (Pett et al.), and 2015/259495 (Liu et al.), all of which are incorporated by reference in their entirety herein. In some embodiments, the adhesive also preferably has good release from a liner that will be used on the rear (or back or bottom) major surface of the backing.

In some embodiments, the adhesive is a pressure-sensitive adhesive (PSA). Some exemplary suitable classes of pressure-sensitive adhesives include polyacrylate adhesives, polyalphaolefin adhesives, polyvinyl acrylates, rubber resin adhesives, silicone adhesives, polydiorganosiloxane polyurea compolymers, mixtures or the like. Some exemplary suitable rubber resin adhesives include those formed using a tackified elastomer where a preferred elastomer is an A-B type block copolymer wherein the A blocks and B blocks are configured in linear (e.g. diblock or triblock copolymer), radial or star configurations. The A block can be formed of a mono-alkenylarene, preferably a polystyrene block having a molecular weight between 4000 and 50,000, preferably between 7000 and 30,000. The A block content is preferably about 10 to 50 weight percent, preferably about 10 to 30 weight percent of the block copolymer. Other exemplary suitable A blocks may be formed from alpha-methylstyrene, t-butyl-styrene and other ring alkylated styrenes, as well as mixtures thereof. The B block may be formed of an elastomeric conjugated diene, generally polyisoprene, polybutadiene or copolymers thereof having an average molecular weight from about 5000 to about 500,000, preferably from about 50,000 to about 200,000. The B block dienes can also be hydrogenated. In some embodiments, the B block content is generally 90 to 50 percent, preferably 90 to 70 percent by weight.

The pressure-sensitive adhesives or adhesive fibers can be mixed with particulates, such as sorbent particulate material, fumed silica, carbon black, glass beads, glass bubbles, clay particles, metal particles, and the like. Tackifiers (solid or liquid), plasticizers, colorants, end block resins, oils, cross-linkers, etc. may be included. Fillers, plasticizers, and other property modifiers, such as flow modifiers, dyes, pigments, flame retardants, stabilizers, antioxidants, compatibilizers, antimicrobial agents, electrical conductors, and thermal conductors, may be incorporated in the pressure-sensitive adhesive composition.

In some embodiments, the adhesive layer is applied to the entire rear (or back or bottom) major surface of the backing. In some embodiments, the adhesive does not cover the entire rear (or back or bottom) major surface of the backing. In some embodiments, the adhesive covers at least 50% of the backing, or at least 75%, or at least 90%, or at least 95% of the total surface of the rear (or back or bottom) major surface of the backing. In some embodiments, the adhesive is pattern coated onto the rear (or back or bottom) major surface of the backing. In some embodiments, the adhesive is coated on (for example, blowing on or roll coating), sprayed on, or laminated to the backing.

In some embodiments, the backing and adhesive form a conjugate multicomponent system, as described in, for example, U.S. Pat. No. 6,107,219 (Joseph et al.), incorporated by reference herein in its entirety. In such embodiments, the adhesive component layer or region and non-adhesive component layer or region are present in separate distinct regions in a conjugate multicomponent fiber. For example, multicomponent fiber layers or regions can be in the form of two, or more, overlaying layered fibers, sheath-core or concentric layered fiber arrangements or in “island in the sea” type fiber layer structures. One component region would comprise the adhesive component layer or region and a second component region would comprise the non-adhesive material layer or region. Generally the adhesive fiber component region will provide at least a portion of the exposed outer surface of the multicomponent conjugate fiber. Preferably, the individual components of the multicomponent conjugate fibers will be present substantially continuously along the fiber length in discreet zones, which zones preferably extend along the entire length of the fibers.

In some embodiments, the backing and adhesive combination is breathable and/or porous. In some embodiments, the backing and adhesive combination is highly breathable and/or porous, making the support article comfortable to wear and/or to minimize or prevent itching, irritation, or undesirable skin reactions. In some embodiments, the backing and adhesive combination allows for moisture release. The more porous and/or breathable the backing and adhesive combination, the better the support article will release moisture caused by sweating or being worn in water or humid environments. In some embodiments, the backing and adhesive combination has a breathability and/or porosity of between about 6 and about 20 mm H₂O measured using the pressure drop test. In some embodiments, the backing layer has a breathability and/or porosity of between about 10 and about 20 mm H₂O measured using the pressure drop test. In some embodiments, the backing+adhesive combination has a breathability and/or porosity of at least about 6 mm H₂O measured using the pressure drop test. In some embodiments, the backing+adhesive combination has a breathability of at least about 10 mm H₂O measured using the pressure drop test. The breathability and/or porosity was measured using the pressure drop test described above.

In some embodiments, the backing and adhesive combination has a cross-directional tensile strength of between about 3 lbf (13.3 N) and about 11 lbf (48.9 N). In some embodiments, the backing and adhesive combination has a cross-directional tensile strength of between about 4 and about 10 lbf (44.5 N). In some embodiments, the backing and adhesive combination has a cross-directional tensile strength of greater than about 3 lbf (13.3 N). Tensile strength can be measured as described above.

In some embodiments, the backing and adhesive combination has a machine-directional tensile strength of between about 4 lbf (17.8 N) and about 15 lbf (66.7 N). In some embodiments, the backing and adhesive combination has a machine-directional tensile strength of between about 5 lbf (22.2 N) and about 13 lbf (57.8 N). In some embodiments, the backing and adhesive combination has a machine-directional tensile strength of greater than about 4 lbf (17.8 N). In some embodiments, the backing and adhesive combination has a machine-directional tensile strength of greater than about 6 lbf (26.7 N). In some embodiments, the backing and adhesive combination has a machine-directional tensile strength of less than about 15 lbf (66.7 N). In some embodiments, the backing and adhesive combination has a machine-directional tensile strength of less than about 12 lbf (53.4 N). Tensile strength can be measured as described above.

In some embodiments, the backing and adhesive combination has a cross-directional elongation at break of about 900%. In some embodiments, the backing and adhesive combination has a cross-directional elongation at break of between about 600% and about 900%, or about 600% and about 800%. Elongation at break was measured as described herein.

In some embodiments, the backing and adhesive combination has a machine-directional elongation at break of about 1000%. In some embodiments, the backing and adhesive combination has a machine-directional elongation of between about 350% and about 1000%, or between about 450-550%. Elongation was measured as described herein. Elongation at break was measured as described herein.

Adhesive Used to Adhere the Reinforcing Portion(s), Bump(s), or Strap(s) to the Backing:

Any of the adhesives described above or herein can be used to adhere the reinforcing portion(s), bump(s), or strap(s) to the backing. Where a different adhesive is used to adhere the reinforcing portion(s), bump(s), or strap(s) to the backing, the adhesive can be any desired adhesive and need not be capable of use on skin since it would not necessarily be used directly on human skin. Additionally or alternatively, the adhesive need not have the porosity or breathability described above and/or herein. Additionally or alternatively, the adhesive need not have the same release properties from a liner described above and/or herein, since the adhesive may not be in contact with the liner. In some embodiments, the adhesive used to adhere the reinforcing portion(s), bump(s), or strap(s) to the backing may adhere more strongly than the adhesive used to adhere the support article to the user. Some exemplary suitable classes of pressure-sensitive adhesives include polyacrylate adhesives, polyalphaolefin adhesives, polyvinyl acrylates, rubber resin adhesives, silicone adhesives, polydiorganosiloxane polyurea compolymers, mixtures or the like.

Reinforcing Portion(s)

The reinforcing portion(s) can have a size, shape, thickness, material, etc. that allow the reinforcing portion(s) to be rigid enough to provide support, compression, and/or pain relief, conformable enough to permit ease of movement and comfort, and/or thin enough to provide a discreet material whose presence is not readily detectable under clothing. In some embodiments, it is preferred that the reinforcing portion not snap or break during use, which often happens with some existing braces that include plastic reinforcement materials. Any reinforcing portion that provides these qualities may be used. In some embodiments, the shape and/or size of the reinforcing portion is tailored for use on a specific area or region of the body. Exemplary reinforcing portion shapes include, but are not limited to, almond shapes, ellipses, ovals, circles, hemispheres, quadrilaterals, hexagons, heptagons, any shapes shown in the Figures of the present disclosure, etc. In some embodiments, the reinforcing portion(s) do not have adhesive on the user skin-facing major surface. In some embodiments, the reinforcing portion(s) have adhesive on the user skin-facing major surface.

In some embodiments, the reinforcing portion is positioned between the backing and the user's skin, when the support article is in use. In some embodiments, the reinforcing portion is on the front (or top) major surface of the backing instead of being between the backing and the user's skin. For purposes of clarity, none of the figures show these embodiments, but any of the embodiments shown or described herein can include the reinforcing portion on the top (front) major surface of the backing.

In some embodiments, the reinforcing portion is a foam layer. In some embodiments, the reinforcing portion is a shaped memory foam layer. In some embodiments, the reinforcing portion is a shaped memory polymer such as, for example, those described in U.S. Patent Publication No. 2010/155998 (Rule et al.), the entirety of which is incorporated herein. In some embodiments, the foam layer includes at least one of polyethylene (“PE”), cross-linked PE, polyurethane, reticulated (open cell) foam, unreticulated (closed cell) foam, neoprene, melamine, vinyl nitrile, PET, XPS (extruded 1-polystyrene), EPS (expanded polystyrene), phenolic, EPP (expanded polypropylene), and EPE (expanded polyethylene). In some embodiments, the reinforcing portion includes a foam layer as described in U.S. Provisional Patent Application No. 62/429,401 (Young et al.), assigned to the present assignee, the entirety of which is incorporated herein.

In some embodiments, the reinforcing portion includes a multilayer construction. In some embodiments, the multilayer construction includes an adhesive layer between first and second foam layers and that construction between first and second skin layers. In other words, the outermost layer is a first skin layer adjacent to a first foam layer adjacent to an adhesive layer adjacent to a second foam layer adjacent to an outer second skin layer. In some embodiments, one or more of the foam layers include microspheres or expandable microspheres. In some embodiments, the microspheres are at least partially embedded in the adhesive layer. In some embodiments, at least one of the skin layers is substantially free of microspheres. In some embodiments, both skin layers are substantially free of microspheres. In some embodiments, the expandable microspheres in one or more of the foam layer are homogenously distributed throughout the foam layer. In some embodiments, the expandable microspheres in one or both of the foam layers are present in a range of between about 4 wt % to about 35 wt % or about 5 wt % to about 25 wt %, based on the total weight of the respective first and second pluralities of expandable microspheres.

Where present, the microspheres may be compressible and retain their spherical shape and integrity after compression, resulting in the reinforcing portion being very resilient and having a high restoration force. During use/wear of the support article, the reinforcing portion(s) readily conform to user movement but continue to provide support because the expanded spheres are resilient and provide strength and support before, during, and after compression. The force exerted to restore the support article is greater than a traditional open cell foam because the microspheres are compressed and have a higher restoration force vs. traditional foam. The microsphere foam is unique in that the more microspheres are added the higher the restoration force due to the increase in number of microspheres present. In a traditional open or closed cell foam a decrease in density typically results in a decreased restoration force because there is a reduction in the amount of polymer present. In the microsphere foam the shell of the microspheres provides a large part of the restoration force—in a traditional foam the polymer itself provides the restoration force—lower density traditional foam=less polymer=lower restoration force.

In some embodiments, the expandable microsphere loading is between about 3% and about 50%. In some embodiments, the microsphere loading is greater than about 3% or greater than about 5%, or greater than about 7%. In some embodiments, the microsphere loading is less than about 50% or less than about 45%, or less than about 40%, or less than about 35%, or less than about 25%, or less than about 15%, or less than about 10%. This resulted in a lightweight, conformable (compressible) foam structure with reduced density loading.

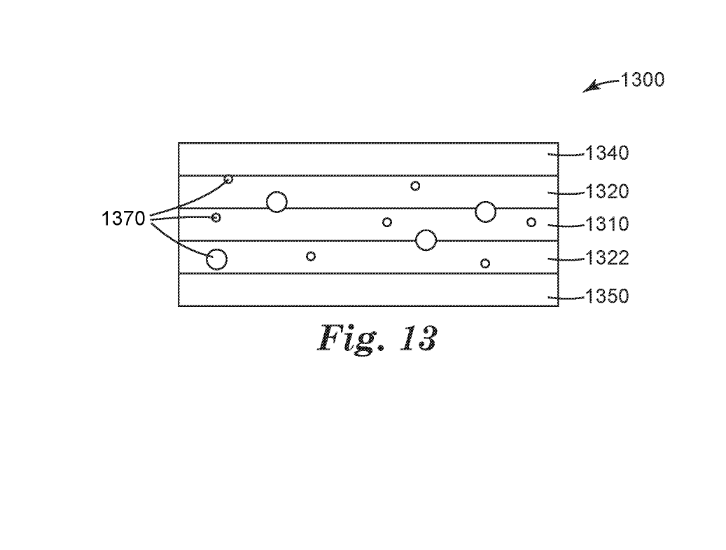

A schematic cross section of an exemplary construction as described above is shown in FIG. 13 in which reinforcing portion 1300 includes an adhesive layer 1310 whose first and second major surfaces 1312, 1314 are each adjacent to first and second foam layers 1320, 1322. First foam layer 1320 is adjacent to a first skin layer 1340, and second foam layer 1322 is adjacent to a second skin layer 1350. First and second skin layers 1340 and 1350 give reinforcing portion 1300 rigidity, while first and second foam layers 1320, 1322 provide softness, resiliency, and conformability. Microspheres 1370 are in at least some of the adhesive layer 1310, first foam layer 1320, and/or second foam layer 1322. More information about each layer of these embodiments of the reinforcing portion is below.

Skin Layer(s): Exemplary skin layers include, for example, at least one of a polyethylene, a polypropylene, a polyurethane, a polylactic acid, ethylene and methacrylate ester copolymer, or copolymers thereof. In some embodiments, at least one of the skin layers includes at least one of a polyolefinic material (e.g., polypropylene and/or polyethylene), modified polyolefinic material, polyvinyl chloride, polycarbonate, polystyrene, polyester (including co-polyester), polylactide, polyvinylidene fluoride, (meth)acrylic (e.g., polymethyl methacrylate), urethane, acrylic urethane, ethylene vinyl acetate copolymer, acrylate-modified ethylene vinyl acetate polymer, ethylene acrylic acid copolymers, nylon, engineering polymer (e.g., a polyketone and/or polymethylpentane), or elastomer (e.g., natural rubber; synthetic rubber; styrene block copolymer containing isoprene, butadiene, or ethylene (butylene) blocks; metallocene-catalyzed polyolefin, polyurethanes; or polydiorganosiloxane). First and second skin layers can be the same or different in composition, density, thickness, etc.

In some embodiments, the use of skins to increase stiffness is highly effective due to the fact that the stiffness of a material increases with the cube of the thickness. In some embodiments, strategically placing the skins on the outer portion of the foam layers provides high relative stiffness in a thin layer while retaining the compression and resilience provided by a foam. If the entire structure were foam without skins, the thickness of the total reinforcing portions could be significantly greater. In some embodiments, combining a resilient, compressible foam with non-foamed (or minimally foamed) skin layer(s) results in a thinner reinforcing portion than of comparable modulus vs. that of a monolithic foamed layer. In some embodiments, the skins also provide tear strength to the foamed layer.

Foam Layer(s):

Exemplary foam layers include, for example, at least one of a polyethylene, a polyurethane, a polylactic acid, a polypropylene, an ethylene and methacrylate ester copolymer, or copolymers thereof. First and second foam layers can be the same or different in composition, density, thickness, etc.

Adhesive Layer:

The adhesive can be any one of a number of pressure sensitive adhesives or non-pressure sensitive adhesive. Examples of suitable pressure sensitive adhesives include a natural rubber-based adhesive, a synthetic rubber based adhesive, a styrene block copolymer-based adhesive, a polyvinyl ether-based adhesive, a poly(methyl acrylate)-based adhesive, a polyolefin-based adhesive, or a silicone-based adhesive. As used herein, an adhesive that is “based” on a particular component means that the adhesive includes at least 50 wt. % of the particular component, based on the total weight of the adhesive. An exemplary adhesive is available under the designated trade designation “KRATON MD6748” from PolyOne.

Suitable non-pressure sensitive adhesives include those that “self-bond” or “block” at the temperature at which the polymeric multilayer material is extruded. Examples of suitable non-pressure sensitive adhesives include very low density polyethylene resins or ethylene copolymer resins with high comonomer content such as a high vinyl acetate containing ethylene vinyl acetate resin.

In some embodiments, the one or more reinforcing portions have a Shore A durometer hardness of between about 10 and about 100. In some embodiments, the one or more reinforcing portions have a Shore A durometer hardness of at least about 10, or at least about 20, or at least about 30, or at least about 40, or at least about 50. In some embodiments, the one or more reinforcing portions have a Shore A durometer hardness of less than about 100, or less than about 90, or less than about 80. In some embodiments, the one or more reinforcing portions have a Shore A durometer hardness of between about 50 and about 80. Shore A durometer hardness can be measured according to ASTM D2240 (2000) using testing equipment commercially obtained as “MODEL #8 SHORE A AND MODEL #9 SHORE D” from Pacific Transducer Corp (PTC Instruments), Los Angeles, Calif.

In some embodiments, the one or more reinforcing portions have a Shore D durometer hardness of between about 10 and about 60. In some embodiments, the one or more reinforcing portions have a Shore D durometer hardness of at least about 10, or at least about 20, or at least about 30. In some embodiments, the one or more reinforcing portions have a Shore D durometer hardness of less than about 60, or less than about 50. In some embodiments, the one or more reinforcing portions have a Shore D durometer hardness of between about 30 and about 50. Shore D durometer hardness can measured according to ASTM D2240 (2000) using testing equipment commercially obtained as “MODEL #8 SHORE A AND MODEL #9 SHORE D” from Pacific Transducer Corp (PTC Instruments), Los Angeles, Calif.

In some embodiments, the reinforcing portion(s) has a cross-directional tensile strength of between about 13 lbf (57.8 N) and about 28 lbf (129.0 N). In some embodiments, the reinforcing portion(s) has a cross-directional tensile strength of greater than about 13 lbf (57.8 N) or greater than about 15 lbf (66.7 N). In some embodiments, the reinforcing portion(s) has a cross-directional tensile strength of less than about 28 lbf (129.0 N) or less than about 25 lbf (111.2 N). Tensile strength is measured as described herein.

In some embodiments, the reinforcing portion(s) has a machine-directional tensile strength of between about 16 lbf (71.2 N) and about 31 lbf (137.9 N). In some embodiments, the reinforcing portion(s) has a machine-directional tensile strength of between about 18 lbf (80.1 N) and about 28 lbf (124.6 N). In some embodiments, the reinforcing portion(s) has a machine-directional tensile strength of greater than about 16 lbf (71.2 N), or greater than about 20 lbf (89.0 N). In some embodiments, the reinforcing portion(s) has a machine-directional tensile strength of less than about 31 lbf (137.9 N) or less than about 28 lbf (124.6 N). Tensile strength is measured as described herein.

In some embodiments, the reinforcing portion is relatively thin or low profile compared to a brace. In some embodiments, the reinforcing portion has a thickness of less than 2 cm (787 mil), or less than 1.5 cm (591 mil), or less than 1 cm (394 mil), or less than 0.5 cm (197 mil), or less than 0.25 cm (98 mil). In some embodiments, the reinforcing portion is 100 mil (0.254 cm), or less than 90 mil (0.229 cm), or less than 80 mil (0.203 cm), or less than 70 mil (0.178 cm), or less than 60 mil (0.152 cm), or less than 50 mil (0.127 cm), or less than 40 mil (0.102 cm), or less than 30 mil (0.076 cm), or less than 20 mil (0.051 cm). In some embodiments, the reinforcing portion has a thickness of at least 20 mil (0.051 cm), at least 30 mil (0.076 cm), or at least 50 mil (0.127 cm). In some embodiments, the reinforcing portion has a thickness of between about 0.025 cm (9.8 mil) to about 0.155 cm (61 mil).

In some embodiments, the reinforcing portion has a density of between about 0.20 g/cm³ to about 0.60 g/cm³, or in some embodiments, 0.25 g/cm³ to 0.4 g/cm³.

In some embodiments, the reinforcing portion(s) is generally non-elastic. In some embodiments, the reinforcing portion(s) have an elongation at break of between about 10% and about 50%, or between 15% and about 25%.

In some embodiments, the support article and/or reinforcing portion(s) has a compression of at least 40 mm Hg, or at least 41 mm Hg, or in some embodiments, 42 mm Hg. Compression can be measured as is described herein.

In some embodiments, the reinforcing portion(s) is generally non-elastic. In some embodiments, the reinforcing portion(s) have an elongation at break of between about 10% and about 50%, or between 15% and about 25%.

In some embodiments, the reinforcing portion(s) are relatively non-absorbent. A reinforcing portion is considered relatively non-absorbent if, after submersion of a 10 gram sample of the reinforcing portion in water at room temperature for 24 hours followed by removing any visible traces of water from the exterior surface of the sample with a tissue, the mass of the sample increases by less than 20% by weight. In some embodiments the mass of such a sample may increase by less than 10% by weight, less 5% by weight, or even less than 1% by weight.

In some embodiments, the reinforcing portion covers at least 10% of the total surface area of the backing, or at least 15, or at least 20%. In some embodiments, the reinforcing portion covers no greater than 75% of the total surface area of the backing, or no greater than 70, or no greater than 60%.

Release Liner

Some embodiments of the present disclosure also include a release liner. Commercially available release liners that can be used include, for example, Polyslik®, PrimeLiner®, Film Plus®, or Lopasil® from Loparex, Cary, N.C.; and CF2 from The Griffin Network, Fallsington, Pa.

Bump

The bump can be any desired size, shape, thickness, etc that permits it to assist in providing targeted, localized contact and/or pressure to the affected area. Exemplary reinforcing shapes include, but are not limited to, almond shapes, ellipses, ovals, circles, hemispheres, quadrilaterals, hexagons, heptagons, top hat, cylindrical, flat top, concave top, convex top, square, rectangular, etc. The sides of the bump can be rounded or straight. Rounded side bumps will generally lack corners and thus may be desirable in some application. Those bumps with sides may include, for example, tapered sides. A variety of exemplary bump shapes and sizes are shown in FIGS. 8-13, each of which is a perspective schematic view of an exemplary bump for use with any of the support articles described herein. Some commercially available bumps are those sold as 3M Bumpon™ protective products, and of which can be used with the support articles described herein.

In some embodiments, the bump includes or is made of a polymer, a plastic, a layered nonwoven or woven, foam, gel pad, air pillow, metal, wood. In some embodiments, the bump(s) are polyurethane and their rear surface is coated with a pressure sensitive adhesive. Urethane can be a desired composition in some application because it is a durable, resilient elastomer. Further, urethane provides long aging resiliency (it will not crack or harden over time), good abrasion resistance, and resists marring or staining during use. In some embodiments, the pressure sensitive adhesive includes at least one of natural rubber, synthetic rubber, acrylic, or silicone. The bump can be colored or clear. Where the bump is colored, it may be, for example, skin colored, white, black, brown, gray, etc. In some embodiments, the bump is transparent. In some embodiments, the bump is colored and transparent.

In some embodiments, the bump has a height (or thickness projecting from the backing) of between about 1.0 mm and about 40.0 mm, more preferably between about 5.0 mm and about 20 mm, or between about 2 mm and about 10 mm. In some embodiments, the height is greater than 1.0 mm, or greater than 5.0 mm, or greater than 10.0 mm, or greater than 15.0 mm. In some embodiments, the bump has a height of less than about 40 mm, less than about 20 mm, less than about 15 mm, or less than about 10 mm. All dimensions are measured without an adhesive liner on the bump.

In some embodiments, the bump has a width of between about 2 mm and about 50 mm, or between about 5 mm and about 30 mm, or between about 8 mm and about 20 mm. In some embodiments, the bump has a width of greater than about 2 mm, or about 5 mm, or about 8 mm, or about 10 mm. In some embodiments, the bump has a width of less than about 50 mm, or 40 mm, or 30 mm, or 20 mm, or 15 mm. All dimensions are measured without an adhesive liner on the bump.

In some embodiments, the bump has a size that is between about 10% and about 80% of the total area of the backing. In some embodiments, the bump has a size that is between about 15% and about 50% of the total area of the backing.

In some embodiments, the bump has a Shore M hardness of between about 70 and 80, or about 72 and 75. Shore M hardness is measured according to ASTM D2240. In some embodiments, the bump has a resilience percent of between about 3% and about 35%, or between about 5% and about 30%. Resilience is measured according to ASTM-D2632 with a 0.125 inch sample. In some embodiments, the bump has a tensile strength of between about 550 and about 750 lbs/in^{t} or between about 4 and 5.3 MPa². Tensile strength of the bump can be measured according to ASTM-D412, Die A. In some embodiments, the bump has excellent 90 degree peel adhesion. To obtain maximum surface adhesion, the surface to which the bump is applied is preferably at least one of unified, dry, and free of contaminants when the bump is applied. During application, the bump is preferably applied with some pressure and allowed time (dwell) to dry or set before use or further manufacturing.

Some embodiments have a single bump. Some embodiments have more than one bump. The bumps in embodiments having more than one bump can have different sizes, shapes, thicknesses, materials, etc.

Strap:

The strap can be made of any material that can stretch and/or extend by at least about 50%. In some embodiments, the strap is made of the same material as the backing. In some embodiments, the strap is made of a different material than the backing. Some exemplary materials for the strap include, for example, polyolefinic material (e.g., polypropylene and/or polyethylene), modified polyolefinic material, polyvinyl chloride, polycarbonate, polystyrene, polyester (including co-polyester), polylactide, polyvinylidene fluoride, (meth)acrylic (e.g., polymethyl methacrylate), urethane, acrylic urethane, ethylene vinyl acetate copolymer, acrylate-modified ethylene vinyl acetate polymer, ethylene acrylic acid copolymers, nylon, engineering polymer (e.g., a polyketone and/or polymethylpentane), or elastomer (e.g., natural rubber; synthetic rubber; styrene block copolymer containing isoprene, butadiene, or ethylene (butylene) blocks; metallocene-catalyzed polyolefin, polyurethanes; or polydiorganosiloxane). In some embodiments, the strap provides customizable compression.

In some embodiments, a portion of the strap is attached or adhered to a portion of the backing. In some embodiments where the strap is adhered, the adhesive is any adhesive that securely affixes the strap to the backing. Some exemplary adhesives include any of the adhesives described herein. Some additional exemplary adhesives include, for example, acrylate, natural rubber-based adhesive, a synthetic rubber based adhesive, a styrene block copolymer-based adhesive, a polyvinyl ether-based adhesive, a poly(methyl acrylate)-based adhesive, a polyolefin-based adhesive, or a silicone-based adhesive.

In some embodiments where a portion of the strap is mechanically attached or affixed to a portion of the backing, the strap can be attached by ultrasonic welding, extrusion, co-extrusion, RF welding, hook and loop, etc.

Exemplary straps are shown and described in U.S. Provisional Application Nos. (attorney docket no. 79039US002 and 79039US003), the entirety of each of which are incorporated by reference herein.

Support Article:

In some embodiments, the support article is a patch, cover, sheet, or strip. In some embodiments, the support article is water resistant. In some embodiments, the support article can be worn for multiple days. In some embodiments, the support article can be worn for up to three (3) days. In some embodiments, the support article will not come off when exposed to humid environments or activities including, for example, in the shower, in humid environments, during exposure to sweat, and/or while swimming. In some embodiments, the support article is easy to apply and/or easy to remove. In some embodiments, the support article provides compression.

In some embodiments, the support article is a patch, cove, sheet, or strip. In some embodiments, the support article is water resistant. In some embodiments, the support article can be worn for multiple days. In some embodiments, the support article can be worn for up to three (3) days. In some embodiments, the support article will not come off when exposed to humid environments or activities including, for example, in the shower, in humid environments, during exposure to sweat, and/or while swimming. In some embodiments, the support article is easy to apply and/or easy to remove. In some embodiments, the support article provides compression.

In some embodiments, the support article is at least some of lightweight and/or comfortable to wear. In some embodiments, the support article has a weight of between about 20 gsm and about 500 gsm. In some embodiments, the support article has a weight of between about 30 gsm and about 300 gsm).

In some embodiments, the support article is conformable. In some embodiments, the support articles have a conformability of less than 13 inches (33.0 cm). In some embodiments, the support articles have a conformability of greater than about 8 inches (20.2 cm).

In some embodiments, the support article is low profile. In some embodiments, the support article (including the backing, adhesive, and reinforcing portion(s)) minus the bump has a thickness of between about 10 mil (0.025 cm) and about 500 mil (1.27 cm). As stated herein, the bump can be any desired size and thickness to provide the desired pressure.

In some embodiments, the support article includes at least one of a hot/cold formulation and/or a pain-reducing medication (e.g., lidocaine) as part of the support article. In some embodiments, the support article further includes a medicament. In some embodiments, the medicament is one of pain-reducing agent and/or an agent that provides for heating or cooling relative to body temperature (or the sensation of heating or cooling relative to body temperature).

In some embodiments, the medicament, hot/cold formulation, and/or a pain-reducing medication is applied to the reinforcing portion and/or to the adhesive that holds the backing adjacent to the user's skin. In such embodiments, it may be desirable that the reinforcing portion has minimal porosity and/or breathability to ensure that the hot/cold formulation and/or a pain-reducing medication only contacts the affected area of the user.

In some embodiments, the support article (or a portion thereof, such as the reinforcing portion) includes only a single plane of symmetry in the major surface. In some embodiments, the support article (or a portion thereof, such as a reinforcing portion) has rounded perimeters without straight regions and/or corners.

In some embodiments, the support article includes at least one of a hot/cold formulation and/or a pain-reducing medication (e.g., lidocaine) as part of the support article. In some embodiments, the support article further includes a medicament. In some embodiments, the medicament is one of pain-reducing agent and/or an agent that provides for heating or cooling relative to body temperature (or the sensation of heating or cooling relative to body temperature).

In some embodiments, the hot/cold formulation and/or a pain-reducing medication is applied to the reinforcing portion and/or to the adhesive that holds the backing adjacent to the user's skin. In such embodiments, it may be desirable that the reinforcing portion has minimal porosity and/or breathability to ensure that the hot/cold formulation and/or a pain-reducing medication only contacts the affected area of the user.

In some embodiments, the support article (or a portion thereof, such as the reinforcing portion) includes only a single plane of symmetry in the major surface. In some embodiments, the support article (or a portion thereof, such as a reinforcing portion) has rounded perimeters without straight regions and/or corners.

The following examples describe some exemplary constructions of various embodiments of the support articles and methods of making the support articles described in the present application. The following examples describe some exemplary constructions and methods of constructing various embodiments within the scope of the present application. The following examples are intended to be illustrative, but are not intended to limit the scope of the present application.

### EXAMPLES

All parts, percentages, ratios, etc. in the examples and the rest of the specification are by weight, unless noted otherwise. Solvents and other reagents used were obtained from Sigma-Aldrich Chemical Company; Milwaukee, Wis. unless otherwise noted. The following abbreviations are used: cm=centimeters; mm=millimeters; in =inch; m=meters; RPM=revolutions per minute; kg=kilograms; oz=ounces; lb=pounds; Pa=Pascals; min=minutes; hr=hours; gsm=grams per square meter (g/m²); DW=downweb (or alternatively MD=machine direction); CW=crossweb; lbf=pound-force; and N=Newton. The terms “weight %”, “% by weight”, and “wt %” are used interchangeably.

Test Methods

Tensile and Elongation Test

Tensile testing and percent elongation tests were performed using a commercially available tensile tester (obtained under the trade designation “ZWICK” from Zwick USA, Kennesaw, Ga.). Tensile and percent elongation testing was performed in the downweb (machine) direction (DW) and in the crossweb (transverse) direction (CW) for all test specimens. Test specimens were cut to be 1 inch (2.54 cm) in width (or in the instance of certain commercially available tapes obtained in 1 inch widths, such tapes were used directly, with the 1 inch width as the crossweb direction). Except where noted, the gauge length was 2 inches (5.08 cm) for downweb testing and 1 inch (2.54 cm) for crossweb testing. A 200 N load cell was used. Depending on the value measured (e.g, downweb or crossweb tensile strength) clamps were attached to downweb ends or crossweb ends of the specimen as appropriate. Crosshead speed was 10 inches/min (25.4 cm/min). For both downweb and crossweb specimens, the maximum force achieved during the test (i.e., tensile strength at break), and the strain at the force maximum (i.e., percent elongation at break), was recorded. Test specimens were tested in triplicate in each of the two principal directions and the results averaged for each of the two principal directions.

Hardness Test

The Shore Durometer Hardness Test (ASTM D-2240 (2000)) was used to measure hardness of foamed specimens (i.e., reinforcing portions). Using testing equipment commercially obtained as “MODEL #8 SHORE A AND MODEL #9 SHORE D” from Pacific Transducer Corp (PTC Instruments), Los Angeles, Calif.

Both Shore A and Shore D hardness were measured. Each Shore tester (SHORE A and SHORE D) was calibrated. To calibrate each tester, a dial on a gauge of each tester was moved to a zero position. From there a probe of each tester was pressed to a surface of a known hardness. The SHORE A tester was considered calibrated if the gauge read 55 and the SHORE D tester considered calibrated if the gauge read 28.

For each specimen, a 1 inch (2.54 cm) square was cut, and the specimen placed on a hard benchtop surface. The specimens were flattened on the surface and a probe of the respective testers was positioned over the specimen. The probe of the instrument was pressed to the specimen, and the equipment provided a readout on the gauge. In each instance, five test specimens were tested and the readouts were averaged. According to section 9.3 within test method D-2240, durometer readings below 20 or above 90 are not considered reliable. By this criterion, some of the test specimens were “too soft.”

Density Test

A pycnometer was used to measure the density of each foamed specimen (i.e., reinforcing portion). The buoyancy force was measured according to ASTM D3575-14 (2014) (“Suffix W—test method B), using a pycnometer (obtained under the trade designation “DELTA RANGE” (Model AG204) from Mettler-Toledo, LLC, Columbus, Ohio). The density was then calculated using Archimedes' principal. That is, specimens were cut from the foam film and first weighed dry (m_{dry}). The specimens were then placed underwater (de-ionized water) to measure the buoyant force (m_{buoyant}) on the pycnometer. Using the formula below, and knowing the density of water is 1 g/cm³, the density of the foamed specimen was calculated.

$${ρ}_{foam} = {ρ}_{water} ( \frac{{m}_{dry}}{{m}_{dry} - {m}_{buoyant}} )$$

Test specimens were tested in triplicate and the results averaged.

Conformability Test

Conformability of foamed specimens (i.e., reinforcing portions) was evaluated as follows. Rectangular test strip specimens 1 inch (2.54 cm) in width and 13 inches (33.02 cm) in length were cut from the foamed material to be tested. The test strip specimens were cut so that the length of the rectangular shaped specimen was in the downweb direction. Where the cut test strip specimens displayed initial curvature, the specimens were flattened by either allowing them to relax on a hard, flat surface for 1 day or by hand manipulation (e.g., by running a finger down the length of the specimen putting slight pressure opposite the curve). The major face of the flattened specimen was then draped over a 26-inch (66.04 cm) circumference heat-capable cylindrical roll of a commercially available laminator (Orca III Laminator available from GBC Pro-Tech, De Forest, Wis.), such that the specimen extended 50% of the circumference of the heat-capable roll (i.e., orthogonal to the length of the roll). The specimen was held in place by taping the ends of the specimen to the heat-capable roll with masking tape. In instances where the roll was heated, the roll was allowed to stabilize at the desired temperature prior to applying the specimen on the roll, and after applying the specimen to the roll, the specimen was allowed to equilibrate on the roll for a predetermined dwell time (0.5 minutes, 1 minute, 5 minutes, or 10 minutes). After the predetermined dwell time, the tape was removed and the specimen was removed from the roll, taking special care not to deform the resultant U-shaped curved specimen. The curved specimen was placed on a hard benchtop surface at ambient temperature (˜−70° F.), with the bottom center of the U-shaped curve contacting the bench top and the specimen ends pointing upward toward the ceiling. The linear distance (in inches) between the two ends of the test specimen was measured using a ruler at various relaxation times, the relaxation time starting from when the specimen was removed from the roll. Higher conformability values indicate lower conformability (e.g., a conformability value of 13 inches after a short relaxation time for a 13-inch test specimen indicates that test specimen was relatively rigid, having relaxed back to original flat form in a short period of time). Reported conformability results are an average of three measurements.

### Examples 1A, 1B, and 1C

An adhesive, a tackified polyacrylate-based non-woven BMF-PSA, was prepared substantially as described at column 18, lines 16-29 (Adhesive Sample 2) of U.S. Pat. No. 6,107,219 (Joseph et al.), onto a conventional polycoated liner. The basis weight of the adhesive was about 60 gsm (about 1.5 mils (0.04 mm) in thickness). A non-woven backing was prepared substantially as described at column 19, lines 48-55 (Backing Samples 8-10) of U.S. Pat. No. 6,107,219, except that HL-2812 PT (an extrudable grade permanent pressure sensitive hot melt adhesive available from H.B. Fuller, Minneapolis, Minn.) was used rather than KRATON PSA, and the backing further contained 3 wt % of a tan pigment (comprised of pre-blended polyurethane (80%)/pigment (20%) available as Product No. FBPUR2103-85-AE TAN from Clariant, Minneapolis, Minn., as described at column 18 lines 55-63 of U.S. Pat. No. 6,107,219 (Joseph et al.).

Three different non-woven backings were prepared at three different basis weights: Example 1A had a basis weight of about 100 gsm (about 11.75 mils (0.298 mm) in thickness); Example 1B had a basis weight of about 150 gsm (about 14.17 mils (0.360 mm) in thickness); and Example 1C had a basis weight of about 200 gsm (about 18.42 mils (0.468 mm) in thickness). Each of the non-woven backings were laminated to the adhesive using a laminator, substantially as described at column 22, lines 1-11 (Example 1) of U.S. Pat. No. 6,107,219. The resulting adhesive-coated backings had the following total basis weights (backing with adhesive, excluding the liner): Example 1A: about 160 gsm (about 13.25 mils (0.337 mm) in total thickness, excluding the liner); Example 1B: about 210 gsm (about 15.75 mils (0.400 mm) in total thickness, excluding the liner); and Example 1C: about 260 gsm (about 19.92 mils (0.506 mm) in total thickness, excluding the liner).

The tensile strength and elongation of the adhesive-coated backing (liner free) in each of the down web and cross web directions as measured according to the Tensile and Elongation Test and results are presented in Table 1 below. Table 1 further includes comparative tensile strength and elongation data for commercially available tapes and wraps.

TABLE 1
Down Web and Cross Web Tensile Strength and Elongation
Percent for Various Examples and Comparative Examples.

| | | Tensile Strength | Elongation |
|---|---|---|---|
| | | (lbf) | (%) |
| Example 1A | DW (2-inch gauge length) | 5.39 (24.0 N) | 470.94 |
| | CW (1-inch gauge length) | 4.26 (18.9 N) | 648.32 |
| Example 1B | DW (2-inch gauge length) | 8.30 (36.9 N) | 522.05 |
| | CW (1-inch gauge length) | 6.24 (27.8 N) | 635.31 |
| Example 1C | DW (2-inch gauge length) | 10.57 (47.02 N) | 496.34 |
| | CW (1-inch gauge length) | 9.53 (42.4 N) | 612.47 |
| Comparative | DW (2-inch gauge length) | 24.63 (109.6 N) | 162.46 |
| Example A | CW (1-inch gauge length) | 36.07 (160.4 N) | 42.58 |
| Comparative | DW (2-inch gauge length) | 11.87 (52.80 N) | 108.29 |
| Example B | CW (1-inch gauge length) | 27.61 (122.8 N) | 19.07 |
| Comparative | DW (2-inch gauge length) | 17.93 (79.76 N) | 229.09 |
| Example C | CW (1-inch gauge length) | 35.92 (159.8 N) | 28.98 |
| Comparative | DW (2-inch gauge length) | 14.76 (65.66 N) | 195.37 |
| Example D | CW (1-inch gauge length) | 7.81 (34.7 N) | 166.27 |
| Comparative | DW (2-inch gauge length) | 20.58 (91.54 N) | 156.65 |
| Example E | CW (1-inch gauge length) | 32.07 (142.7 N) | 43.47 |
| Comparative | DW (2-inch gauge length) | 37.66 (167.5 N) | 451.20 |
| Example F | CW (1-inch gauge length) | 45.35 (201.7 N) | 22.16 |

Comparative Example A: MUELLER KINESIOLOGY TAPE, BLACK 1-STRIP ROLL kinesiology tape from Mueller Sports Medicine (Prairie du Sac, WI).
Comparative Example B: KT TAPE ™ KINESIOLOGY THERAPEUTIC TAPE, ORIGINAL BLACK cotton kinesiology tape from KT Health, LLC (American Fork, Utah).
Comparative Example C: KT TAPE ™ KINESIOLOGY THERAPEUTIC TAPE, PRO PINK synthetic kinesiology tape from KT Health, LLC (American Fork, UT).
Comparative Example D: 3M ™ COBAN ™ self-adherent wrap available from 3M Company (St. Paul, MN).
Comparative Example E: ACE ™ KINESIOLOGY TAPE available from 3M Company (St. Paul, MN).
Comparative Example F: ACE ™ ELASTIC BANDAGE available from 3M Company (St. Paul, MN).

### Example 2

The reinforcing portion (a blown film foam) was prepared as follows. A seven layer film was produced using a seven layer annular stack die (obtained under the trade designation “COEX 7-LAYER” (Type LF-400) from Labtech Engineering, Samutprakarn, Thailand). Airflow to the die was manually controlled to achieve a blow up ratio of about 2:1. The bubble was subsequently collapsed about ten feet above die and rolled up. The feed materials were supplied by 7 independent 20 mm diameter extruders, each with about a 30:1 length to diameter ratio. A first extruder was used to melt and extrude an extrudable pressure sensitive adhesive (obtained under the trade designation “KRATON MD6748” from Polyone, Avon Lake, Ohio) into an inside channel of the annular stack die. A screw speed of 30 revolutions per minute was used. The melt temperature was maintained at 180° C. A second, third, fourth, fifth and sixth extruder were used to feed, to the next five channels of the annular stack die, a blend of an ethylene methyl acrylate (EMA) copolymer (obtained under the trade designation “ELVALOY 1609” from Dupont, Wilmington, Del.) and a masterbatch pellet containing a 65% concentration of an expandable microsphere (obtained under the trade designation “EXPANCEL 950 MB 80” from Akzo Nobel, Amsterdam, Netherlands). The blend ratio was maintained at 92% of the ethylene methyl acrylate and 8% of the microsphere masterbatch. Extruder speeds were maintained at 60 revolutions per minute. A seventh extruder was used to feed a low density polyethylene (LDPE) resin (obtained under the trade designation “PETROTHENE NA217000” from LyondellBasell, Houston, Tex.) to the outside channel of the annular stack die. A melt temperature of 190° C. was maintained. The extruder speed for this resin was maintained at 70 revolutions per minute. Because the bubble was subsequently collapsed, and the innermost layer of the film was a pressure-sensitive adhesive, the finished film, after edge trimming of the collapsed bubble, was in effect a five layer film where the outermost (or “skin”) layers were the LDPE, the center layer was the result of the joining of two layers of the pressure sensitive adhesive, and layers 2 and 4 were each the product of the merging, while in the melt, of five original layers of the EMA with expandable microspheres. The blown film foam produced was 55-60 mils (1.40-1.52 mm) thick and designated as Example 2.

The blown film foam was tested for tensile properties, hardness, and density, as described above. The results are provided in Table 2, below. Additionally, a 118 mil (3.00 mm) thick commercially available foam (obtained under the trade designation “WOODBRIDGE #SM25WH” from Woodbridge Polyurethane, Troy, Mich.), indicated as Comparative Example G in Table 2, was also tested.

TABLE 2
Tensile Properties, Hardness, and Density of Example 2 and Comparative Foam.

| | | Tensile | Elongation | Shore A | Shore D | Density |
|---|---|---|---|---|---|---|
| | | Strength (lbf) | (%) | Hardness | Hardness | (g/cm³) |
| Example 2 | | | | 70 | 38 | 0.41 |
| | DW (2-in gauge length) | 21.6 (96 N) | 22 | | | |
| | CW (1-in gauge length) | 17.8 (79 N) | 16 | | | |
| Comparative | | | | Too Soft | Too Soft | 0.04 |
| Example G | | | | | | |
| | DW (2-in gauge length) | 3.4 (15 N) | 360 | | | |
| | CW (1-in gauge length) | 3.1 (14 N) | 475 | | | |

The blown film foams from Example 2 and Comparative Example G were evaluated according to the Conformability Test, at ambient roll temperature (˜70° F./˜21° C.) and at 90° F. (32° C., simulating human skin temperature) roll temperature, as described above.

Comparative Example H was a flexible stabilizing bar, 7.94 inches (20.2 cm) in length and 0.5 inches (1.27 cm) in width obtained from a commercially available adhesive knee support strip under the trade designation KT FLEX REINFORCED ADHESIVE STRIPS from KT Health, LLC, American Fork, Utah. Comparative Example H was evaluated in a similar fashion as described in the Conformability Test, except that the flexible stabilizing bar was used directly in the test without further modification. Notably, the key component to providing conformability measurement data is the conformability of the reinforcing portion. The adhesive coated backing has a minimal impact on conformability measurement data. The results are provided in Table 3, below.

TABLE 3
Dwell Time, Relaxation Time, and Relaxation Length at
Varying Temperatures.

| | | | Relaxation | Relaxation | |
|---|---|---|---|---|---|
| | Temperature | Dwell Time | Time | Length | |
| | (° F.) | (minutes) | (minutes) | (inches (cm)) | |
| Example 2 | 90 | 0.5 | 0.5 | 12 | (30.5) |
| | 90 | 10 | 0.5 | 11.5 | (29.2) |
| | ~70 | 0.5 | 0.5 | 12.3 | (31.2) |
| | ~70 | 10 | 0.5 | 12 | (30.5) |
| Comparative | 90 | 0.5 | 0.5 | 13 | (33.0) |
| Example G | | | | | |
| | 90 | 10 | 0.5 | 13 | (33.0) |
| | ~70 | 0.5 | 0.5 | 13 | (33.0) |
| | ~70 | 10 | 0.5 | 13 | (33.0) |
| Comparative | 90 | 0.5 | 0.5 | 7.94 | (20.2) |
| Example H | | | | | |
| | 90 | 10 | 0.5 | 7.94 | (20.2) |
| | ~70 | 0.5 | 0.5 | 7.94 | (20.2) |
| | ~70 | 10 | 0.5 | 7.94 | (20.2) |

It is apparent from Table 3 that Comparative Examples G and H were largely non-conformable under the test conditions, with both relaxing to their original length after a relaxation time of only 30 seconds.

### Example 3: Elbow Support Article

An elbow support article as shown in FIGS. 1 and 2B was made by laser cutting a adhesive-coated backing of the shape shown in FIG. 1 from the backing/adhesive combination described in Example 1B. A reinforcing portion as shown in FIG. 2B was laser cut from the foam described in Example 2. The reinforcing portion was then laminated to the adhesive side of the adhesive coated backing.

Next, a hemispherical-shaped polyurethane bump about 0.63 inches (16.0 mm) in diameter and about 0.31 inches (7.9 mm) in height coated with a pressure sensitive adhesive on its flat surface (available from 3M Company (St. Paul, Minn.) under the trade designation 3M BUMPON PROTECTIVE PRODUCTS SJ5027, having a reported Shore A hardness of about 70) was applied to the reinforcing portion by removing the liner from the bump and pressing the adhesive face of the bump onto the center of the reinforcing portion with hand pressure, thus providing the elbow support article as shown in FIGS. 1 and 2B. A conventional liner was then applied to the adhesive face of the article, the conventional liner including a small circularly shaped hole sized slightly larger than the diameter of the bump to allow the bump to protrude through the liner. The length of the adhesive-coating backing in the longest direction was about 5.47 inches (13.9 cm) and the length of the reinforcing portion in the longest direction was about 2.05 inches (5.21 cm). Approximately 89% of the surface area of the skin-facing side of the article was adhesive-coated (with the balance of the surface area due to the non-adhesive reinforcing portion). In a similar fashion, support articles described herein may be prepared.

Reference throughout this specification to “one embodiment,” “some embodiments,” “one or more embodiments” or “an embodiment,” whether or not including the term “exemplary” preceding the term “embodiment,” means that a particular feature, structure, material, or characteristic described in connection with the embodiment is included in at least one embodiment of the certain exemplary embodiments of the present disclosure. Thus, the appearances of the phrases such as “in one or more embodiments,” “in certain embodiments,” “in one embodiment” or “in an embodiment” in various places throughout this specification are not necessarily referring to the same embodiment of the certain exemplary embodiments of the present disclosure. Furthermore, the particular features, structures, materials, or characteristics may be combined in any suitable manner in one or more embodiments.

The recitation of all numerical ranges by endpoint is meant to include all numbers subsumed within the range (i.e., the range 1 to 10 includes, for example, 1, 1.5, 3.33, and 10). Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The polymers described herein can terminate in any suitable way. In some embodiments, the polymers can terminate with an end group that is independently chosen from a suitable polymerization initiator, —H, —OH, a substituted or unsubstituted (C₁-C₂₀) hydrocarbyl (e.g., (C₁-C₁₀)alkyl or (C₆-C₂₀)aryl) interrupted with 0, 1, 2, or 3 groups independently selected from —O—, substituted or unsubstituted —NH—, and —S—, a poly(substituted or unsubstituted (C₁-C₂₀)hydrocarbyloxy), and a poly(substituted or unsubstituted (C₁-C₂₀)hydrocarbylamino).

The terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

All references mentioned herein are hereby incorporated by reference in their entirety.

With reference to the Figures, like numerals are used to designate like components throughout the set of Figures.

Those having skill in the art will appreciate that many changes may be made to the details of the above-described embodiments and implementations without departing from the underlying principles thereof. Further, various modifications and alterations of the present invention will become apparent to those skilled in the art without departing from the spirit and scope of the invention. The scope of the present application should, therefore, be determined only by the following claims and equivalents thereof.

What is claimed is:

1. A support article, comprising:
a backing having a front major surface and a rear major surface;
an adhesive adjacent to or included in at least a portion of the rear major surface of the backing layer; and
a bump that extends or protrudes from the rear major surface of the backing.

2. The support article of claim 1, further comprising:
a release liner adjacent to at least a portion of the adhesive.

3. The support article of claim 1, further comprising:
a strap attached or adhered to the front major surface of the backing, the strap being capable of stretching across at least a portion of the support article.

4. The support article of claim 3, wherein the strap includes at least one of a polyolefin, a modified polyolefin, a polyvinyl chloride, a polycarbonate, polystyrene, polyester, polylactide, polyvinylidene fluoride, (meth)acrylic, urethane, acrylic urethane, ethylene vinyl acetate copolymer, acrylate-modified ethylene vinyl acetate polymer, ethylene acrylic acid copolymers, nylon, engineering polymer, or elastomer.

5. The support article of claim 1, wherein the backing includes at least one of a polyurethane film, a polyethylene film, a polypropylene film, a PVC film, a nonwoven material, and/or a woven material.

6. The support article of claim 1, wherein at least one of the backing or the backing and adhesive combination has a breathability and/porosity of between about 3 and about 12 mm H₂O measured using the pressure drop test.

7. The support article of claim 1, wherein the backing has a weight of between about 25 gsm to about 300 gsm.

8. The support article of claim 1, wherein at least one of the backing or the backing and adhesive combination has a cross-directional tensile strength of between about 4 lbf (17.8 N) and about 9 lbf (40.0 N) and/or a machine-directional tensile strength of between about 5 lbf (22.2 N) and about 10 lbf (44.5 N).

9. The support article of claim 1, wherein at least one of the backing or the backing and adhesive combination has a cross-directional elongation at break of between about 600% and about 900% and/or a machine-directional elongation at break of between about 350% and about 1000%.

10. The support article of claim 1, wherein the backing has a thickness of about 0.01 cm to about 1 cm.

11. (canceled)

12. The support article of claim 1, wherein the backing and adhesive form a conjugate multicomponent system.

13. The support article of claim 1, further comprising:
one or more reinforcing portions adjacent to at least a portion of the rear major surface of the backing or at least a portion of the front major surface of the backing.

14. The support article of claim 13, wherein the one or more reinforcing portions includes at least one of foam or a shaped memory material.

15. The support article of claim 13, wherein the adhesive adheres the one or more reinforcing portions to the front or rear major surface of the backing.

16. The support article of claim 13, wherein the one or more reinforcing portions comprise:
an adhesive layer having first and second major surfaces;
a first foam layer adjacent to first major surface of the adhesive layer;
a second foam layer adjacent to second major surface of the adhesive layer;
a first skin layer adjacent to first foam layer; and
a second skin layer adjacent to second foam layer.

17. The support article of claim 16, further including microspheres in at least one of the first or second foam layers.

18. The support article of claim 13, comprising at least two reinforcing layers that are on layered on one another.

19. The support article of claim 13, wherein at least one of the reinforcing portions is separate from the support article and can be independently applied by the user before the full support article is applied.

20. (canceled)

21. A method of applying a support article, comprising:
removing a liner from a rear major surface of the support article to expose an adhesive;
positioning the support article adjacent to a user's body in a desired location;
applying the support article to the user such that the adhesive contacts the user's skin; and
putting pressure on the support article to cause the adhesive to adhere to the user's skin.

22. The method of claim 21, wherein the support article is the support article of any of claims 1-20.
